SEQUENCE LISTING (9 sequences)

SEQ ID NO: 1 - protein, 12 aa, Homo sapiens
MADNFSLHDALS

SEQ ID NO: 2 - protein, 14 aa, Homo sapiens
MADNFSLHDALSGS

SEQ ID NO: 3 - protein, 10 aa, Homo sapiens
WGNQPAGAGG

SEQ ID NO: 4 - protein, 16 aa, Homo sapiens
YPGAPGAYPGAPAPGV

SEQ ID NO: 5 - protein, 11 aa, Homo sapiens
GNPNPQGWPGA

SEQ ID NO: 6 - protein, 12 aa, Homo sapiens
YPSSGQPSATGA

SEQ ID NO: 7 - protein, 18 aa, Homo sapiens
YPGQAPPGAYPGQAPPGA

SEQ ID NO: 8 - protein, 19 aa, Homo sapiens
YPGAPAPGVYPGPPSGPGA

SEQ ID NO: 9 - protein, 12 aa, Homo sapiens
YPSSGQPSATGA

(19) United States
(12) Patent Application Publication
(10) Pub. No.: US 2012/0220671 A1
(43) Pub. Date: Aug. 30, 2012

(54) USE OF GALECTIN-3 FOR DETECTING AND PROGNOSING HEART FAILURE AFTER ACUTE CORONARY SYNDROME

(75) Inventor: Pieter Muntendam, Boxford, MA (US)

(73) Assignee: BG Medicine, Inc., Waltham, MA (US)

(21) Appl. No.: 13/362,804

(22) Filed: Jan. 31, 2012

Related U.S. Application Data
(60) Provisional application No. 61/438,245, filed on Jan. 31, 2011.

(51) Int. Cl. G01N 33/566; A61K 35/00; G01N 21/25; G01N 21/64; C12Q 1/06

(52) U.S. Cl. 514/789; 436/501; 435/7.92; 435/39; 435/7.94

(57) ABSTRACT

The present invention relates to materials and methods for predicting the risk of heart failure in a subject with Acute Coronary Syndrome. The invention further relates to monitoring the efficacy of treatment for heart failure in a subject with Acute Coronary Syndrome.

### REFERENCE TO RELATED APPLICATIONS

This application claims the benefit of and priority to U.S. Provisional Patent Application No. 61/438,245, filed Jan. 31, 2011, the complete disclosure of which is incorporated by reference herein.

### BACKGROUND

Galectin-3 is a β-galactoside-binding lectin implicated in cardiac fibrosis and remodeling, elevated in models of failure-prone hypertrophied hearts, and with prognostic value in patients with heart failure (HF). (See, e.g., U.S. Patent Publication No. US 2008-0193954 A1.)

### SUMMARY OF THE INVENTION

It has now been discovered that concentrations of the human protein galectin-3 in body fluids can be used to predict or monitor disease progression or therapeutic efficacy in patients with Acute Coronary Syndrome (ACS). An ACS patient's galectin-3 blood concentration can be determined to evaluate risk for developing heart failure. In addition, an ACS patient's galectin-3 blood concentration can be monitored over the course of a treatment for heart failure to determine the efficacy of treatment.

In one aspect, the invention relates to a method of diagnosing the risk of heart failure development after an acute coronary syndrome in a subject. The method can include measuring the level of galectin-3 in a sample from an subject, comparing the level of galectin-3 to a reference value, and determining whether the level of galectin-3 identifies a likelihood that the subject will develop heart failure. In some embodiments, the reference value for galectin-3 is in the range from about 10.0 ng/mL to about 25.0 ng/mL. In other embodiments, the reference value for galectin-3 is in the range from about 13.0 ng/mL to about 19.2 ng/mL. Some embodiments include identifying the subject as having an increased likelihood of developing heart failure if the galectin-3 value is greater than a reference value, and identifying the subject as having a decreased likelihood of developing heart failure if the galectin-3 value is less than the reference value. In some embodiments, the subject is a human subject.

The method can include the step of transmitting, displaying, storing, or printing; or outputting to a user interface device, a computer readable storage medium, a local computer system or a remote computer system, information related to the likelihood of developing heart failure in the subject. The measured galectin-3 levels can be determined by at least one of an immunoassay, a colorimetric assay, a turbidimetric assay, and flow cytometry.

In another aspect, the invention relates to a method of assessing a subject after an acute coronary syndrome for risk of developing heart failure, the method comprising: measuring a galectin-3 blood concentration in a sample from the subject to determine the presence or absence of a galectin-3 blood concentration indicative of risk of developing heart failure.

In other aspects, the invention relates to a method of monitoring development or progression of heart failure in a subject after an acute coronary syndrome, including measuring a galectin-3 blood concentration in a sample from the subject to determine the presence or absence of a galectin-3 blood concentration indicative of the development or progression of heart disease.

In some embodiments, the sample comprises blood, serum or plasma.

The method can include treating a patient after an acute coronary syndrome by initiating a heart failure therapy in a patient having a determined galectin-3 blood concentration indicative of risk of heart failure. Some embodiments include the additional step of monitoring the patient's galectin-3 blood concentration after the therapy is initiated.

In a selected or treated patient, the blood concentration of galectin-3 may be determined to be above a minimum threshold, below a maximum threshold or within a target range defined by a minimum and a maximum threshold. The minimum threshold may be, for example, more than 10 ng/ml; between 10 and 15 ng/ml; between 15 and 20 ng/ml; between 20 and 25 ng/ml; between 25 and 30 ng/ml; or be more than 30 ng/ml. The maximum threshold may be, for example, below 70 ng/ml; below 60 ng/ml; below 40 ng/ml; between 30 and 40 ng/ml; between 25 and 30 ng/ml; between 20 and 25 ng/ml; or between 15 and 20 ng/ml.

### DETAILED DESCRIPTION OF THE INVENTION

The terms “heart failure,” “HF,” “congestive heart failure,” or “CHF” as used herein, refer to the complex clinical syndrome that impairs the ability of the ventricle to fill with or eject blood. Any structural or functional cardiac disorder can cause HF, with the majority of HF patients having impaired left ventricular (LV) myocardial function. Symptoms of HF include dyspnea (shortness of breath), fatigue, and fluid retention. The American Heart Association (AHA) has identified 4 stages in the progression or development of HF. Patients in stages A and B show clear risk factors but have not yet developed HF. Patients in stages C and D currently exhibit or in the past have exhibited symptoms of HF. For example, Stage A patients are those with risk factors such as coronary artery disease, hypertension or diabetes mellitus who do not show impaired left ventricular (LV) function. Stage B patients are asymptomatic, but have cardiac structural abnormalities or remodeling, such as impaired LV function, hypertrophy or geometric chamber distortion. Stage C patients have cardiac abnormalities and are symptomatic. Stage D patients have refractory HF in which they exhibit symptoms despite maximal medical treatment. They are typically recurrently hospitalized or unable to leave the hospital without specialized intervention.

Galectin-3 is a structurally unique member of a family of multifunctional β-galactoside-binding lectins (Gabius (2006) Crit. Rev. Immunol. 26:43-79). Expression of galectin-3 has been associated with the epithelium and inflammatory cells including macrophages, neutrophils and mast cells. Galectin-3 has been implicated in a variety of biological processes important in heart failure including myofibroblast proliferation, fibrogenesis, tissue repair, cardiac remodeling, and inflammation (Liu et al. (2009) Am. J. Physiol. Heart Circ. Physiol. 296(2):H404-12; Papaspyridonos et al. (2008) Arterioscler. Thromb. Vasc. Biol. 28(3):433-40; Henderson et al. (2006) Proc. Natl. Acad. Sci. USA 103:5060-5065; Sharma et al. (2004) Circulation 110:3121-3128; Sano et al. (2000) J. Immunol. 165(4):2156-64; Kuwabara et al. (1996) J. Immunol. 156(10):3939-44).

Applicants have developed methods permitting the use of circulating galectin-3 protein levels to predict risk of developing heart failure following Acute Coronary Syndrome (ACS). Knowledge of a patient's galectin-3 level is informative of patient outcome following ACS. Furthermore, detection of galectin-3 to identification of patients at risk for heart failure can occur before symptoms of heart failure appear, which can allow treatment to be administered earlier, and result in a more favorable outcome.

### Galectin-3 Detection

The present invention provides methods for predicting risk of developing heart failure following an Acute Coronary Syndrome (ACS) by measuring the levels of markers such as galectin-3, optionally in combination with one or more other markers (e.g., BNP, NT-proBNP). Many methods for detecting of a protein of interest, with or without quantitation, are well known and can be used in the practice of the present invention. Examples of such assays are described below and can include, for example, immunoassays, chromatographic methods, and mass spectroscopy. Such assays can be performed on any biological sample including, among others, blood, plasma, and serum. Accordingly, multiple assays can be used to detect galectin-3, and samples can be analyzed from one or more sources.

Markers can be detected or quantified in a sample with the help of one or more separation methods. For example, suitable separation methods may include a mass spectrometry method, such as electrospray ionization mass spectrometry (ESI-MS), ESI-MS/MS, ESI-MS/(MS)ⁿ (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)ⁿ, or atmospheric pressure photoionization mass spectrometry (APPI-MS), APPI-MS/MS, and APPI-(MS)ⁿ. Other mass spectrometry methods may include, inter alia, quadrupole, fourier transform mass spectrometry (FTMS) and ion trap. Spectrometric techniques that can also be used include resonance spectroscopy and optical spectroscopy.

Other suitable separation methods include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE), or other chromatographic techniques, such as thin-layer, gas or liquid chromatography, or any combination thereof. In one embodiment, the biological sample to be assayed may be fractionated prior to application of the separation method.

Markers can may be detected or quantified by methods that do not require physical separation of the markers themselves. For example, nuclear magnetic resonance (NMR) spectroscopy may be used to resolve a profile of a marker from a complex mixture of molecules. An analogous use of NMR to classify tumors is disclosed in Hagberg (1998) NMR Biomed. 11:148-56, for example.

A marker in a sample also may be detected or quantified, for example, by combining the marker with a binding moiety capable of specifically binding the marker. The binding moiety may include, for example, a member of a ligand-receptor pair, i.e., a pair of molecules capable of having a specific binding interaction. The binding moiety may also include, for example, a member of a specific binding pair, such as antibody-antigen, enzyme-substrate, nucleic acid-nucleic acid, protein-nucleic acid, protein-protein, or other specific binding pairs known in the art. Binding proteins may be designed which have enhanced affinity for a target. Optionally, the binding moiety may be linked with a detectable label, such as an enzymatic, fluorescent, radioactive, phosphorescent or colored particle label. The labeled complex may be detected, e.g., visually or with the aid of a spectrophotometer or other detector, or may be quantified.

Galectin-3 levels can be quantitated by performing an immunoassay. A galectin-3 immunoassay involves contacting a sample from a subject to be tested with an appropriate antibody under conditions such that immunospecific binding can occur if galectin-3 is present, and detecting or measuring the amount of any immunospecific binding by the antibody. Any suitable immunoassay can be used, including, without limitation, competitive and non-competitive assay systems using techniques such as Western blots, radioimmunoassays, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays and protein A immunoassays.

In a “sandwich” assay, two molecules (“binding moieties”) such as monoclonal antibodies that specifically bind to non-overlapping sites (epitopes) on galectin-3 are used. Typically, one binding moiety is immobilized on a solid surface where it binds with and captures galectin-3. This first binding moiety is therefore also referred to as the capture binding moiety. A second binding moiety is detectably labeled, for example, with a fluorophore, enzyme, or colored particle, such that binding of the second binding moiety to the galectin-3-complex indicates that galectin-3 has been captured. The intensity of the signal is proportional to the concentration of galectin-3 in the sample. The second binding moiety is therefore also referred to as the detection binding moiety or label binding moiety. A binding moiety can be any type of molecule, as long as it specifically binds to a portion of the N-terminus of galectin-3. In a preferred embodiment, the binding moieties used are monoclonal anti-galectin-3 antibodies, i.e., monoclonals raised against or otherwise selected to bind to separate portions of galectin-3.

Such assay procedures can be referred to as two-site immunometric assay methods, “sandwich” methods or (when antibodies are the binders) “sandwich immunoassays.” As is known in the art, the capture and detection antibodies can be contacted with the test sample simultaneously or sequentially. Sequential methods can be accomplished by incubating the capture antibody with the sample, and adding the labeled detection antibody at a predetermined time thereafter (sometimes referred to as the “forward” method). Alternatively, the labeled detection antibody can be incubated with the sample first and then the sample can be exposed to the capture antibody (sometimes referred to as the “reverse” method). After any necessary incubation(s), which may be of short duration, to complete the assay, the label is measured. Such assays may be implemented in many specific formats known to those of skill in the art, including through use of various high throughput clinical laboratory analyzers or with a point of care or home testing device.

In one embodiment, a lateral flow device may be used in the sandwich format wherein the presence of galectin-3 above a baseline sensitivity level in a biological sample will permit formation of a sandwich interaction upstream of or at the capture zone in the lateral flow assay. See, for example, U.S. Pat. No. 6,485,982. The capture zone may contain capture binding moieties such as antibody molecules, suitable for capturing galectin-3, or immobilized avidin or the like for capture of a biotinylated complex. See, for example, U.S. Pat. No. 6,319,676. The device may also incorporate a luminescent label suitable for capture in the capture zone, the concentration of galectin-3 being proportional to the intensity of the signal at the capture site. Suitable labels include fluorescent labels immobilized on polystyrene microspheres. Colored particles also may be used.

Other assay formats that may be used in the methods of the invention include, but are not limited to, flow-through devices. See, for example, U.S. Pat. No. 4,632,901. In a flow-through assay, one binding moiety (for example, an antibody) is immobilized to a defined area on a membrane surface. This membrane is then overlaid on an absorbent layer that acts as a reservoir to pump sample volume through the device. Following immobilization, the remaining protein-binding sites on the membrane are blocked to minimize non-specific interactions. In operation, a biological sample is added to the membrane and filters through the matrix, allowing any analyte specific to the antibody in the sample to bind to the immobilized antibody. In a second step, a labeled secondary antibody may be added or released that reacts with captured marker to complete the sandwich. Alternatively, the secondary antibody can be mixed with the sample and added in a single step. If galectin-3 is present, a colored spot develops on the surface of the membrane.

The most common enzyme immunoassay is the “Enzyme-Linked Immunosorbent Assay (ELISA).” ELISA is a technique for detecting and measuring the concentration of an antigen using a labeled (e.g., enzyme-linked) form of the antibody. There are different forms of ELISA, which are well known to those skilled in the art. Standard ELISA techniques are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984), J. Clin. Chem. Clin. Biochem. 22:895-904. A preferred enzyme-linked immunosorbent assay kit (ELISA) for detecting galectin-3 is commercially available (BG Medicine, Waltham, Mass.).

In a “sandwich ELISA,” an antibody (e.g., anti-galectin-3) is linked to a solid phase (i.e., a microtiter plate) and exposed to a biological sample containing antigen (e.g., galectin-3). The solid phase is then washed to remove unbound antigen. A labeled antibody (e.g., enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be measured. Any of the immunoassays described herein suitable for use with the kits and methods of the present invention can also use any binding moiety in the place of an antibody.

A detailed review of immunological assay design, theory and protocols can be found in numerous texts in the art, including Butt, W. R., Practical Immunology, ed. Marcel Dekker, New York (1984) and Harlow et al. Antibodies, A Laboratory Approach, ed. Cold Spring Harbor Laboratory (1988).

In general, immunoassay design considerations include preparation of antibodies (e.g., monoclonal or polyclonal antibodies) having sufficiently high binding specificity for the target to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the target. The higher the antibody binding specificity, the lower the target concentration that can be detected. As used herein, the terms “specific binding” or “binding specifically” are understood to mean that the binding moiety, for example, a binding protein, has a binding affinity for the target of greater than about 10⁵ M⁻¹, more preferably greater than about 10⁷ M⁻¹.

Antibodies to an isolated target marker which are useful in assays for detecting heart failure in an individual may be generated using standard immunological procedures well known and described in the art. See, for example Practical Immunology, supra. Briefly, an isolated marker is used to raise antibodies in a xenogeneic host, such as a mouse, goat or other suitable mammal. The marker is combined with a suitable adjuvant capable of enhancing antibody production in the host, and is injected into the host, for example, by intraperitoneal administration. Any adjuvant suitable for stimulating the host's immune response may be used. A commonly used adjuvant is Freund's complete adjuvant (an emulsion comprising killed and dried microbial cells and available from, for example, Calbiochem Corp., San Diego, or Gibco, Grand Island, N.Y.). Where multiple antigen injections are desired, the subsequent injections may comprise the antigen in combination with an incomplete adjuvant (e.g., cell-free emulsion). Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target and have the desired binding affinity.

Exemplary epitopes from the N-terminus of galectin-3 include, but are not limited to, MADNFSLHDALS (SEQ ID NO:1); MADNFSLHDALSGS (SEQ ID NO:2); WGNQPAGAGG (SEQ ID NO:3); YPGAPGAYPGAPAPGV (SEQ ID NO:4); GNPNPQGWPGA (SEQ ID NO:5); YPSSGQPSATGA (SEQ ID NO:6); YPGQAPPGAYPGQAPPGA (SEQ ID NO:7); YPGAPAPGVYPGPPSGPGA (SEQ ID NO:8); and YPSSGQPSATGA (SEQ ID NO:9). Other galectin-3 epitopes, including non-linear epitopes, can also be used as targets for detection by an anti-galectin-3 antibody. Exemplary antibodies are discussed in U.S. Patent Publication No. 2010/014954, the entire contents of which are incorporated herein by reference.

Antibody binding domains also may be produced biosynthetically and the amino acid sequence of the binding domain manipulated to enhance binding affinity with a preferred epitope on the target. Specific antibody methodologies are well understood and described in the literature. A more detailed description of their preparation can be found, for example, in Practical Immunology, (supra).

In addition, genetically engineered biosynthetic antibody binding sites, also known in the art as BABS or sFv's, may be used to determine if a sample contains a marker. Methods for making and using BABS comprising (i) non-covalently associated or disulfide bonded synthetic V_{H} and V_{L} dimers, (ii) covalently linked V_{H}-V_{L} single chain binding sites, (iii) individual V_{H} or V_{L} domains, or (iv) single chain antibody binding sites are disclosed, for example, in U.S. Pat. Nos. 5,091,513; 5,132,405; 4,704,692; and 4,946,778. Furthermore, BABS having requisite specificity for the marker can be derived by phage antibody cloning from combinatorial gene libraries (see, for example, Clackson et al. Nature 352: 624-628 (1991)). Briefly, phages, each expressing on their coat surfaces BABS having immunoglobulin variable regions encoded by variable region gene sequences derived from mice pre-immunized with an isolated marker, or a fragment thereof, are screened for binding activity against the immobilized marker. Phages which bind to the immobilized marker are harvested and the gene encoding the BABS is sequenced. The resulting nucleic acid sequences encoding the BABS of interest then may be expressed in conventional expression systems to produce the BABS protein.

Multimarker analysis can be used to improve the accuracy of diagnosis and monitoring. For example, blood concentrations of galectin-3 (Gal-3) and brain natriuretic peptide (BNP) can be used to diagnose heart failure and to predict the long-term outcome of heart failure (van Kimmenade et al., J. Am. Coll. Cardiol., 48:1217-24 (2006); Sharma et al., Circulation, 110:3121-28 (2004); Lok et al., Eur. Heart J., 28:141, Abstract 1035 (2007)). BNP and its cleavage equivalent amino-terminal proBNP (NT-proBNP) are elevated in heart muscle and in blood during heart failure as a result of high filling pressures of heart chambers and the stretch of cardiac muscle fibers, and has recently been shown to be predictive of heart failure in patients with ACS (Scirica et al., (2006) “Intensive Statin Therapy and the Risk of Hospitalization for Heart Failure After an Acute Coronary Syndrome in the PROVE IT-TIMI 22 Study,” Journal of the American College of Cardiology, 47(11):2326-2331). Other secondary markers that could be used to diagnose heart failure may include non-polypeptidic cardiac markers such as sphingolipid, sphingosine, sphingosine-1-phosphate, dihydrosphingosine and sphingosylphosphorylcholine (see U.S. Pat. No. 6,534,322). When measuring the levels of the above markers, corrections for age and gender may be incorporated to improve the accuracy of diagnosis.

### EXAMPLES

### Example 1

### Galectin-3 is Associated with the Risk of Developing Heart Failure Following ACS

In a nested case-control study among patients with Acute Coronary Syndrome (ACS) in the Pravastatin or Atorvastatin Evaluation and Infection Trial—Thrombolysis In Myocardial Infarction 22 (PROVE IT-TIMI 22) study, 100 cases were identified with a hospitalization for new or worsening heart failure. Controls (1:1) were matched for age, sex, ACS type, and randomized treatment. Serum galectin-3 (BG Medicine, Inc., Waltham, Mass.) was measured at baseline (7d post ACS). Further procedural details are found in Cannon et al, (2004) “Intensive versus Moderate Lipid Lowering with Statins after Acute Coronary Syndromes,” N Engl J Med. 350(15):1495-1504, and in the correction found at N Engl J Med. (2006); 354:778, each of which is incorporated by reference herein for all purposes.

Patients who developed heart failure had higher baseline galectin-3, median 16.7 ng/ml (interquartile range (IQR): 14.0-20.6) vs 14.6 (IQR: 12.0-17.6), p=0.004. Patients with baseline galectin-3 above the median, 15.6 ng/mL, were twice as likely to develop HF, OR 2.1 (95% Confidence Interval (CI): 1.2-3.6), p=0.01. Galectin-3 showed a graded relationship with risk of heart failure (Table 1). Cases were more likely to have hypertension (HTN), diabetes mellitus (DM), and prior heart failure; after adjustment for these factors, this graded relationship with galectin-3 quartile and heart failure remained significant, adjusted odds ratio (adj-OR) 1.4 (95% CI 1.1-1.9; p=0.02). Each standard deviation (SD) increase in galectin-3 was associated with a 48% increase in the adjusted relative odds of heart failure (p=0.04). These data show that galectin-3 is associated with the risk of developing heart failure following ACS.

Table 1 shows the relative odds of developing heart failure in patients after an Acute Coronary Syndrome, which increases with increased galectin-3 serum concentration.

TABLE 1
Odds ratio for developing heart failure, by galectin-3
concentration category.

| | Galectin-3 category | | | |
|---|---|---|---|---|
| | | | 15.6-19.2 | |
| | <13.0 ng/mL | 13.0-15.5 ng/mL | ng/mL | >19.2 ng/mL |
| Odds ratio | 1.0 | 2.0 | 2.6 | 3.9 |
| (p-value) | (referent) | (p = 0.10) | (p = 0.03) | (p = 0.004) |

The lowest galectin-3 category, <13.0 ng/mL, is the reference category. P=0.003 for trend of odds ratio across categories.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of diagnosing the risk of heart failure development after an acute coronary syndrome in a subject, the method comprising:
(a) measuring the level of galectin-3 in a sample from an subject;
(b) comparing the level of galectin-3 to a reference value;
(c) determining whether the level of galectin-3 identifies a likelihood that the subject will develop heart failure.

2. The method of claim 1, wherein the reference value for galectin-3 is in the range from about 10.0 ng/mL to about 25.0 ng/mL.

3. The method of claim 2, wherein the reference value for galectin-3 is in the range from about 13.0 ng/mL to about 19.2 ng/mL.

4. The method of claim 1, comprising identifying the subject as having an increased likelihood of developing heart failure if the galectin-3 value is greater than a reference value, and identifying the subject as having a decreased likelihood of developing heart failure if the galectin-3 value is less than the reference value.

5. The method of claim 1, comprising the step of transmitting, displaying, storing, or printing; or outputting to a user interface device, a computer readable storage medium, a local computer system or a remote computer system, information related to the likelihood of developing heart failure in the subject.

6. The method of claim 1, wherein the measured galectin-3 levels are determined by at least one of an immunoassay, a colorimetric assay, a turbidimetric assay, and flow cytometry.

7. The method of claim 1, wherein the sample comprises blood, serum or plasma.

8. The method of claim 1, wherein the subject is human.

9. A method of assessing a subject after an acute coronary syndrome for risk of developing heart failure, the method comprising: measuring a galectin-3 blood concentration in a sample from the subject, thereby to determine the presence or absence of a galectin-3 blood concentration indicative of risk of developing heart failure.

10. A method of monitoring development or progression of heart failure in a subject after an acute coronary syndrome, the method comprising measuring a galectin-3 blood concentration in a sample from the subject, thereby to determine the presence or absence of a galectin-3 blood concentration indicative of the development or progression of heart disease.

11. The method of claim 9, wherein the sample comprises blood, serum or plasma.

12. A method of treating a patient after an acute coronary syndrome comprising initiating a heart failure therapy in the patient with acute coronary syndrome having a determined galectin-3 blood concentration indicative of risk of heart failure.

13. The method of claim 12 comprising the additional step of monitoring the patient's galectin-3 blood concentration after the therapy is initiated.

14. The method of claim 9, wherein the patient has a galectin-3 blood concentration determined to be within a target range.

15. The method of claim 9, wherein the patient has a galectin-3 blood concentration determined to be above a minimum threshold.

16. The method of claim 15, wherein the minimum threshold is more than 10 ng/ml.

17. The method of claim 15, wherein the minimum threshold is between 10 and 15 ng/ml.

18. The method of claim 15, wherein the minimum threshold is between 15 and 20 ng/ml.

19. The method of claim 15, wherein the minimum threshold is between 20 and 25 ng/ml.

20. The method of claim 15, wherein the minimum threshold is between 25 and 30 ng/ml.

21. The method of claim 15, wherein the minimum threshold is more than 30 ng/ml.

22. The method of claim 9, wherein the patient has a galectin-3 blood concentration determined to be below a maximum threshold.

23. The method of claim 22, wherein the maximum threshold is below 70 ng/ml.

24. The method of claim 22, wherein the maximum threshold is below 60 ng/ml.

25. The method of claim 22, wherein the maximum threshold is below 40 ng/ml.

26. The method of claim 22, wherein the maximum threshold is between 30 and 40 ng/ml.

27. The method of claim 22, wherein the maximum threshold is between 25 and 30 ng/ml.

28. The method of claim 22, wherein the maximum threshold is between 20 and 25 ng/ml.

29. The method of claim 22, wherein the maximum threshold is between 15 and 20 ng/ml.
